(19) United States
(12) Patent Application Publication
(10) Pub. No.: US 2025/0228874 A1
(43) Pub. Date: Jul. 17, 2025

(54) CALCITRIOL OR CALCITRIOL ANALOGUES FOR TREATING PERIPERHAL NEUROPATHIC PAIN DISORDERS

(71) Applicant: EPTIVA THERAPEUTICS LTD., Oxford (GB)

(72) Inventor: Mark J. FIELD, Great Dunmow (GB)

(21) Appl. No.: 18/697,693

(22) PCT Filed: Oct. 8, 2022
(86) PCT No.: PCT/IB2022/059655

§ 371 Date: Apr. 1, 2024

Related U.S. Application Data
(60) Provisional application No. 63/253,617, filed on Oct. 8, 2021.

(51) Int. Cl. A61K 31/593; A61K 9/00

(52) U.S. Cl. CPC A61K 31/593; A61K 9/0014

(57) ABSTRACT

Disclosed are methods for the treatment of peripheral neuropathic pain disorders associated with keratin dysregulation via localized, topical administration of calcitriol and/or calcitriol analogues. Such keratin-related peripheral neuropathic pain disorders include pachyonychia congenita, Olmsted syndrome, and other disorders involving palmoplantar keratoderma (PPK). A topical treatment composition including a therapeutic amount of calcitriol or an analogue such as cholecalciferol can provide analgesic effects and/or can reduce excessive keratinocyte proliferation.

### CROSS-REFERENCE TO RELATED APPLICATIONS

This application claims priority to and the benefit of U.S. Provisional Patent Application No. 63/253,617, filed Oct. 8, 2021, the entirety of which is incorporated herein by reference.

### BACKGROUND

### Technical Field

The subject matter relates to the treatment of peripheral neuropathic pain disorders, particularly peripheral neuropathic pain disorders associated with keratin disorders, such as pachyonychia congenita, through topical administration of calcitriol and/or derivatives thereof.

### Related Technology

Chronic pain is a burden to many patients and to society as a whole. It has been suggested that up to 1 in 4 people will suffer a chronic pain condition, creating associated economic costs estimated at $600 billion every year in the U.S. alone. Current treatments are only effective in a minority of patients, and new analgesics have only a 2% chance of clinical success.

Vitamin D is a hormone that is important in numerous physiological and pathophysiological processes of the human body. Vitamin D3 (cholecalciferol) is a steroid hormone which requires metabolism to its active form calcitriol (1,25(OH)2-vitamin D3) to interact with the vitamin D receptor and initiate biological effects. Vitamin D is found in some foods and is produced endogenously in the skin via UV sunlight. Such endogenously formed vitamin D needs to be metabolized in the liver and kidneys to produce its active form calcitriol. Vitamin D can also be metabolized in certain tissues including by keratinocytes locally within the skin.

It is recognized that there is, globally, a high prevalence of vitamin D deficiency, which has been associated with numerous conditions such as osteoporosis, diabetes, autoimmune disease, renal diseases, and cancer. Vitamin D3 (cholecalciferol) has been used as a therapeutic supplement. Its active metabolite, calcitriol has been developed as an FDA approved medicinal product for psoriasis.

### SUMMARY

The present disclosure relates to methods for the treatment of peripheral neuropathic pain disorders. Specifically, the present disclosure describes various embodiments of using calcitriol and/or calcitriol analogues to treat peripheral neuropathic pain disorders, particularly keratin related peripheral neuropathic pain disorders such as pachyonychia congenita, Olmsted syndrome, and other disorders involving palmoplantar keratoderma (PPK). Using a network biology approach, the inventor discovered that the biological actions and second messenger pathways affected by calcitriol have similarities to those implicated in the local mediation of pain from keratinocytes. This led to the conclusion that localized action of the active form of vitamin D (calcitriol), and/or an analogue/derivative such as calcipotriol, will have analgesic effects and can provide effective treatment of peripheral neuropathic pain conditions associated with keratin disorders.

The present disclosure further discloses methods of using topical calcitriol compositions in treating peripheral neuropathic pain disorders, particularly keratin related peripheral neuropathic pain disorders such as pachyonychia congenita, Olmsted syndrome, and/or other disorders involving PPK. For example, a method described herein comprises administering a topical composition (e.g., cream, ointment, lotion, spray, balm, salve, oil, gel, etc.) including approximately 0.005% to 0.1% calcitriol (and/or one or more calcitriol analogues) to a patient that has such a condition. In another example, a method described herein comprises administering a dermal patch including calcitriol (and/or one or more calcitriol analogues) to a patient that has such a condition. A topical composition and/or a dermal patch may include cholecalciferol (in addition to or alternative to the calcitriol) which can be locally metabolized to calcitriol in keratinocytes. In one example, a dermal patch may include a concentration of cholecalciferol of 0.0005% to 0.1%.

Patients suffering from pain associated with such keratin disorders generally receive no benefit from conventional analgesics, making the methods of treatment disclosed herein beneficial and useful tools for pain management.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a model of the molecular pathways involved in pachyonychia congenita (see Cao et al., Gene Expression Profiling in Pachyonychia Congenita Skin, J Dermatol Sci. 2015 March; 77(3): 156-165) revised to show possible interaction points at which calcitriol can interact; and

FIGS. 2 and 3 are charts summarizing the results of searches (PubMed and ClinicalTrials.gov, respectively) linking vitamin D, cholecalciferol, and/or calcitriol to specific disease conditions.

### DETAILED DESCRIPTION

### Overview

The present disclosure relates to methods for the treatment of peripheral neuropathic pain disorders. Specifically, the present disclosure describes various embodiments of using calcitriol and/or calcitriol analogues to treat peripheral neuropathic pain disorders, particularly keratin related peripheral neuropathic pain disorders such as pachyonychia congenita, Olmsted syndrome, and other disorders involving PPK. Using a network biology approach, the inventor discovered that several biological actions and second messenger pathways modulated by calcitriol have similarities to those implicated in the local mediation of pain from keratinocytes. This led to the conclusion that localized action of the active form of vitamin D (calcitriol), and/or an analogue/derivative such as calcipotriol, will have analgesic effects and can provide effective treatment of peripheral neuropathic pain conditions associated with keratin disorders.

The present disclosure further discloses methods of using topical calcitriol compositions in treating peripheral neuropathic pain disorders, particularly keratin related peripheral neuropathic pain disorders such as pachyonychia congenita, Olmsted syndrome, and/or other disorders involving PPK. For example, a method described herein comprises administering a topical composition (e.g., cream, ointment, lotion, spray, balm, salve, oil, gel, etc.) including approximately 0.005% to 0.1% calcitriol (and/or one or more calcitriol analogues) to a patient that has such a condition. In another example, a method described herein comprises administering a dermal patch including calcitriol (and/or one or more calcitriol analogues) to a patient that has such a condition. A topical composition and/or a dermal patch may include cholecalciferol (in addition to or alternative to the calcitriol) which can be locally metabolized to calcitriol in keratinocytes. In one example, a dermal patch may include a concentration of cholecalciferol of 0.0005% to 0.1%. Such a dermal patch may additionally include an amount of calcitriol (and/or a calcitriol analogue), or may omit or substantially omit calcitriol and calcitriol analogues.

### Patients/Conditions

PPK is characterized by marked thickening of the epidermis of the skin, particularly on the palms of the hands and soles of the feet. Several disorders involve PPK as a symptom. The condition is believed to be uncurable, and treatments thus focus on symptom management. Unfortunately, pain associated with PPK does not respond well to conventional analgesics.

One example of a condition involving PPK is pachyonychia congenita, where the fingernails and toenails may be misshapen and thickened, in addition to the thickening of the epidermis of the skin. Pachyonychia congenita primarily affects ectoderm-derived epithelial appendages and typically includes oral leukokeratosis, nail dystrophy, and very painful PPK which can often be debilitating. Pachyonychia congenita is caused by genetic mutations in one or more keratin genes. The disease follows an autosomal dominant pattern of inheritance. Thus, on average, half of the children of an affected person will inherit the disorder, regardless of sex.

Because there is no cure for pachyonychia congenita, conventional treatment is broadly aimed at relieving pain symptoms and treating secondary infections. However, cysts, blisters, hyperkeratosis, and other pain-causing effects of the condition do not respond well to conventional analgesic approaches. Moreover, there are at present no licensed treatments specific for the condition.

Keratinocytes produce and metabolize vitamin D locally, which is critical for wound repair. Keratinocytes also metabolize and produce vitamin D throughout the epidermis. Such production and metabolization may occur at various local sites. Keratinocytes work in concert with other epidermal cells (such as Langerhans cells) to regulate the skin.

Olmsted syndrome is another keratin disorder characterized by the presence of PPK. Olmsted syndrome is also known as “mutilating PPK with periorificial keratotic plaques.” Olmsted syndrome most commonly affects the palms, soles, and areas around the eyes and mouth, but can also affect the hair, nails, and even joints. The abnormal skin thickening associated with Olmsted syndrome can cause pain and/or itching and puts the patient at greater risk for skin infections and skin cancer.

### Treatment Compositions

As described herein, although analgesics have conventionally had limited success when used to treat patients having keratin disorders such as discussed above, it has now been discovered that topical administration of calcitriol can beneficially reduce neuropathic pain associated with such keratin disorders and/or reduce keratinocyte proliferation at the local site of application. Keratin disorders such as pachyonychia congenita, Olmsted syndrome, and other PPK-associated conditions can generate porous callouses in patients, which makes affected skin amenable to topical treatments. When calcitriol is included in a composition administered topically to such affected skin, it can beneficially provide localized analgesic effects.

As used herein, concentration percentages refer to weight percentages unless specified otherwise.

The term “calcitriol,” as used herein, is inclusive of analogues/derivatives such as calcipotriol (also known as calcipotriene). Thus, where a method is disclosed in terms of administering calcitriol, the method may include administering calcitriol, analogues/derivatives of calcitriol such as calcipotriol, or combinations thereof. The term 1,25-dihydroxycholecalciferol is used herein to specify the natural form of calcitriol as opposed to an analogue/derivative such as calcipotriol. Treatment compositions may additionally or alternatively include cholecalciferol, which can be locally metabolized to calcitriol by keratinocytes at the site of administration.

In some embodiments, a topical treatment composition containing calcitriol and/or cholecalciferol may be applied to the skin to act on keratinocytes and/or other epidermal cells. In some embodiments, the treatment composition comprises 0.0005% to 0.1% calcitriol and/or cholecalciferol. In some embodiments, the composition can contain 0.001% to 0.05%, or 0.002% to 0.035%, or 0.003% to 0.02%, or 0.004% to 0.01%, or 0.005% calcitriol and/or cholecalciferol, or may include calcitriol and/or cholecalciferol in a concentration range with endpoints defined by any two of the foregoing values. In some embodiments, the administered topical composition can inhibit keratinocyte proliferation at the local site of application.

In some embodiments, the topical composition includes a carrier appropriate for topical administration. The topical composition may be in the form of a cream, ointment, lotion, spray, balm, salve, oil, gel, or other suitable topical form.

Examples of compounds that can be used as or included in carriers include, but are not limited to, water, alcohols (ethanol, isopropyl alcohol, 1,3-propandiol, other polyols), ketones, esters, oils (e.g., mineral oils, essential oils, vegetable, and/or other plant oils), triglycerides, ethers, glycerin, petroleum jelly, waxes, polymers, powders (e.g., talcum powder and/or cornstarch), and surfactants. Gels known in the art can be used as carriers, such as gels containing one or more liquid components together with known gelling agents. In some embodiments, the topical treatment composition can also include one or more adjuvants including, but not limited to, antimicrobial agents, skin conditioners, plant extracts, astringents, moisturizers, emollients, antiseptics, penetration promoters (e.g., dimethyl sulfoxide (DMSO)), and the like.

In some embodiments, the composition is included as part of carrier layer included in a dermal patch. In such embodiments, the carrier layer may comprise natural and/or synthetic polymers. The carrier layer may have sufficient tackiness and/or adhesiveness to enable adherence of the dermal patch to the skin. Such polymers are known in the art and include, for example, polyvinyl pyrrolidone, polysiloxanes, acrylates, methacrylates, and combinations thereof.

### Associated Biological Pathways

Vitamin D deficiencies are related to numerous conditions such as osteoporosis, diabetes, autoimmune diseases, renal diseases, and cancer. However, until now, it was not known that calcitriol could be utilized as a topical analgesic for keratin disorders such as those described herein. The beneficial local analgesic effects of calcitriol are not limited to those with vitamin D deficiencies, but can provide efficacy independent of the patient's systemic vitamin D deficiency status. Potential downstream targets that calcitriol may act on when deployed for pain management include TRPV1, EGFR and TNFα, among others.

At least some of the potential targets identified, such as EGFR, are receptors that play a role in skin growth and/or function. At least some of the potential targets identified, such as TRPV1, are receptors that play a role in the peripheral nervous system. Without being bound to theory, modulation of such receptors upon topical administration of calcitriol may provide a mechanism of action by which the topical calcitriol treats peripheral neuropathic pain and/or reduces excessive keratinocyte proliferation

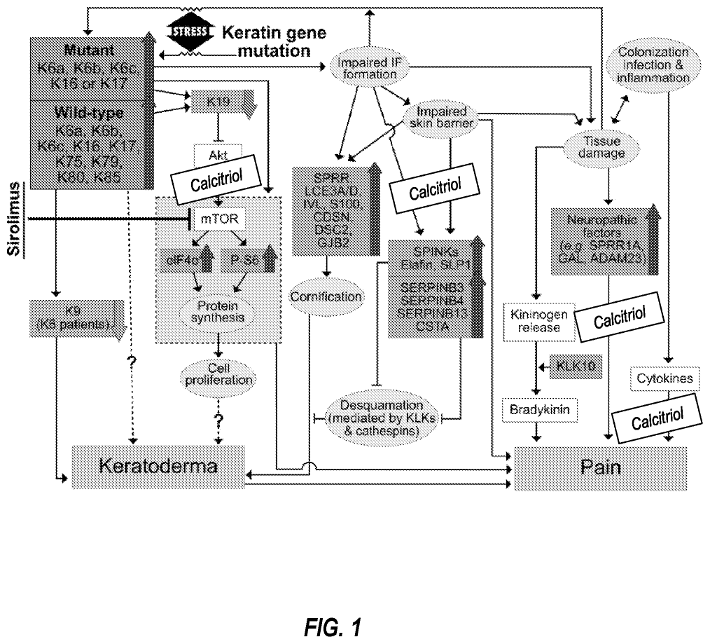

Calcitriol can interact with pachyonychia congenita pathways associated with mTOR, cytokines (e.g., TGF-b, IL1, IL8, IL10), as well as reduce neuropathic factors and promote repair of the skin barrier. FIG. 1 is a model of the molecular pathways involved in pachyonychia congenita (see Cao et al., Gene Expression Profiling in Pachyonychia Congenita Skin, J Dermatol Sci. 2015 March; 77(3): 156-165), revised to show possible interaction points at which calcitriol can interact. For example, calcitriol may interact between phosphate kinase B (Akt) and mTOR, may modulate SPINKs, Elafin, SLP1, SERPINB3, SERPINB4, SERPINB13, and/or CSTA, may modulate production of neuropathic factors such as SPRR1A, GAL, and ADAM23, and/or may modulate production of cytokines.

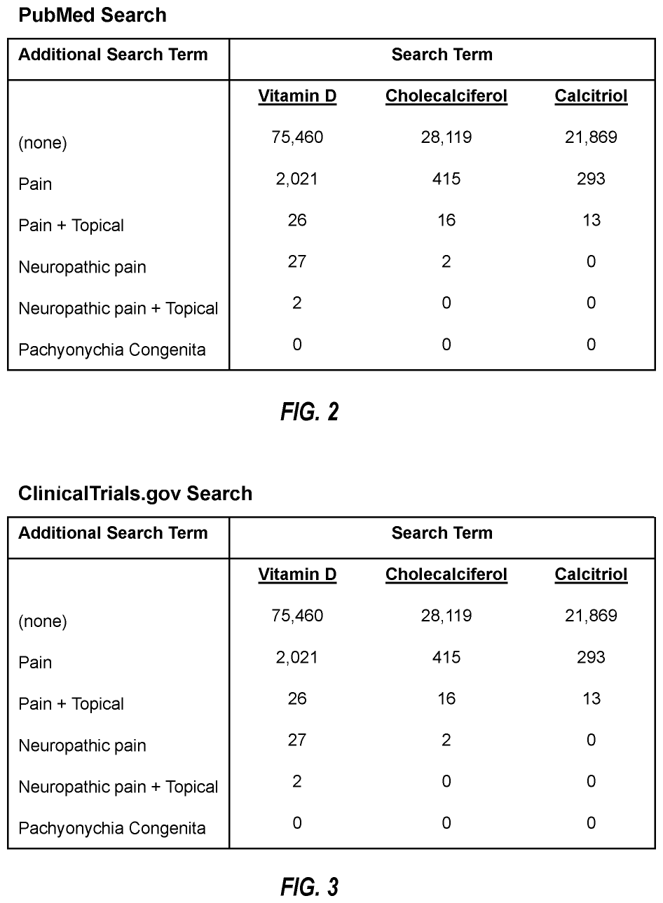

FIGS. 2 and 3 summarize the results of searches (PubMed and ClinicalTrials.gov, respectively) linking vitamin D, cholecalciferol, and/or calcitriol to specific disease conditions. The illustrated charts display an overview of relevant terms searched, either alone or in conjunction with each other, and the resulting number of papers or studies containing those terms. For example, there were no papers containing the terms “calcitriol” and “neuropathic pain” in the results obtained from PubMed (see FIG. 2). Similarly, there were no clinical trials containing both the terms “calcitriol” and “neuropathic pain” in the results obtained from ClinTrials.gov (see FIG. 3).

These results indicate that little to no research has been conducted in investigating the relationship between these compounds and pain, particularly neuropathic pain. The present disclosure beneficially meets a long felt need for providing effective treatment options for patients suffering from keratin disorders such as pachyonychia congenita, Olmsted syndrome, and other disorders involving PPK.

### Additional Terms & Definitions

Although particular examples of treatment compositions and methods are described herein, the examples do not limit the scope of the present disclosure. For example, where specific topical administration forms are described by way of example, it will be understood that other compositions and methods suitable for delivering calcitriol to dermal tissue may additionally or alternatively be used.

Unless otherwise indicated, numbers expressing quantities, proportions, percentages, or other measurements used in the specification and claims are to be understood as optionally being modified by the term “about” or its synonyms, even if the term does not expressly appear. Any numerical range recited herein is intended to include all subranges subsumed therein. When ranges are given, any endpoints of those ranges and/or numbers within those ranges can be combined within the scope of the present disclosure.

Plural use of terms encompasses singular use of the terms and vice versa. For example, while the disclosed coating composition has been described in terms of optionally including “a” carrier or “an” adjuvant, additional carrier components and/or adjuvants may be included.

When the term “about,” “approximately,” “substantially,” “essentially,” or the like are used in conjunction with a stated amount, value, or condition, it may be taken to mean an amount, value, or condition that deviates by 10% or less, 5% or less, 1% or less, 0.1% or less, or 0.01% or less from the stated amount, value, or condition. For example, a recited calcitriol concentration of “about” X percent includes other percentage values that differ from X (higher or lower) by up to 10%, up to 5%, up to 1%, up to 0.1%, or up to 0.01%.

The calcitriol-including treatment compositions disclosed herein should be understood as comprising/including disclosed components, and may therefore include additional components not specifically described. Optionally, a treatment composition as disclosed herein is essentially free or completely free of components that are not specifically described. That is, non-disclosed components may optionally be omitted or essentially omitted from the disclosed treatment composition. For example, a particular adjuvant, vitamin, or carrier component that is not specifically described as being included in the disclosed treatment composition may be optionally excluded (i.e., essentially omitted or completely omitted).

A composition that “essentially omits,” is “essentially free of,” a component may include trace amounts and/or non-functional amounts of the component. For example, the “essentially omitted” component may be included in an amount no more than 10%, no more than 5%, no more than 2.5%, no more than 1%, no more than 0.1%, or no more than 0.01% by total weight of the composition. This is likewise applicable to other negative modifier phrases such as, but not limited to, “essentially omits,” “essentially without,” similar phrases using “substantially” or other synonyms of “essentially,” and the like.

Any headings and subheadings used herein are for organizational purposes only and are not meant to be used to limit the scope of the description or the claims.

### Additional Examples

The disclosed methods and treatment compositions may include, but are not necessarily limited to, features recited in the following clauses:
Clause 1: A method of treating a keratin-related peripheral neuropathic pain disorder, the method comprising: topically administering a treatment composition to a patient suffering from a keratin-related peripheral neuropathic pain disorder, wherein the treatment composition comprises calcitriol and/or cholecalciferol to be locally metabolized to calcitriol.
Clause 2: The method of clause 1, wherein the keratin-related peripheral neuropathic pain disorder involves palmoplantar keratoderma (PPK).
Clause 3: The method of clause 2, wherein the keratin-related peripheral neuropathic pain disorder is pachyonychia congenita.
Clause 4: The method of clause 2, wherein the keratin-related peripheral neuropathic pain disorder is Olmsted syndrome.
Clause 5: The method of any one of clauses 1-4, wherein the administration of the treatment composition provides an analgesic effect to the patient.
Clause 6: The method of any one of clauses 1-5, wherein the administration of the treatment composition reduces keratinocyte proliferation.
Clause 7: The method of any one of clauses 1-6, wherein the treatment composition comprises a cream, ointment, lotion, spray, balm, salve, oil, or gel.
Clause 8: The method of any one of clauses 1-7, wherein the treatment composition comprises cholecalciferol and is associated with a dermal patch configured to be applied to epidermis of the patient.
Clause 9: The method of any one of clauses 1-8, wherein the calcitriol comprises 1,25-dihydroxycholecalciferol, calcipotriol, or both.
Clause 10: The method of any one of clauses 1-9, wherein the treatment composition comprises 0.0005% to 0.1%, or 0.001% to 0.05%, or 0.002% to 0.035%, or 0.003% to 0.02%, or 0.004% to 0.01%, or 0.005% calcitriol and/or cholecalciferol by weight.
Clause 11: A treatment composition for use in topically treating a keratin-related peripheral neuropathic pain disorder according to a method as in any one of clauses 1-10.
Clause 12: Use of a composition as in clause 11 for treating a keratin-related peripheral neuropathic pain disorder.
Clause 13: Use of a composition as in clause 11 in the manufacture of a medicament for the treatment of a keratin-related peripheral neuropathic pain disorder.

1. A method of treating a keratin-related peripheral neuropathic pain disorder, the method comprising:
topically administering a treatment composition to a patient suffering from a keratin-related peripheral neuropathic pain disorder, wherein the treatment composition comprises calcitriol and/or cholecalciferol to be locally metabolized to calcitriol.

2. The method of claim 1, wherein the keratin-related peripheral neuropathic pain disorder involves palmoplantar keratoderma (PPK).

3. The method of claim 2, wherein the keratin-related peripheral neuropathic pain disorder is pachyonychia congenita.

4. The method of claim 2, wherein the keratin-related peripheral neuropathic pain disorder is Olmsted syndrome.

5. The method of claim 1, wherein the administration of the treatment composition provides an analgesic effect to the patient.

6. The method of claim 1, wherein the administration of the treatment composition reduces keratinocyte proliferation.

7. The method of claim 1, wherein the treatment composition comprises a cream, ointment, lotion, spray, balm, salve, oil, or gel.

8. The method of claim 1, wherein the treatment composition comprises cholecalciferol and is associated with a dermal patch configured to be applied to epidermis of the patient.

9. The method of claim 1, wherein the calcitriol comprises 1,25-dihydroxycholecalciferol, calcipotriol, or both.

10. The method of claim 1, wherein the treatment composition comprises 0.0005% to 0.1%, or 0.001% to 0.05%, or 0.002% to 0.035%, or 0.003% to 0.02%, of 0.004% to 0.01%, or 0.005% calcitriol and/or cholecalciferol by weight.

11-21. (canceled)
